(19) United States
(12) Patent Application Publication
(10) Pub. No.: US 2004/0019344 A1
(43) Pub. Date: Jan. 29, 2004

(54) Integrated, one-piece sampling connector unit

(76) Inventors: Grant Peterson, Chicago, IL (US); Armando Bombino, Crystal Lake, IL (US)

Correspondence Address: Michael C. Mayo, Baxter Healthcare Corporation, Law/Transfusion Therapies Division, RLP-30, P.O. Box 490, Rt. 120 and Wilson Road, Round Lake, IL, 60073, US

(21) Appl. No.: 10/607,026

(22) Filed: Jun. 26, 2003

Related U.S. Application Data
(60) Provisional application No. 60/398,913, filed on Jul. 26, 2002.

(51) Int. Cl. A61M 5/32; A61B 5/00

(52) U.S. Cl. 604/411; 600/578

(57) ABSTRACT

A one-piece connector and hub sub-unit for attachment to a sample tube receiver is disclosed. The sub-unit includes a first end defining a first piercing member terminating in a blunt end and a second end adapted for receiving a separate piercing member.

### CROSS REFERENCE TO RELATED APPLICATION

This application claims the benefit of U.S. Provisional Patent Application Serial No. 60/398,913, filed Jul. 26, 2002.

### TECHNICAL FIELD

The present invention relates to methods and apparatus for collecting samples of blood, blood components or other biological fluids. More specifically, the present invention relates to an integrated, one-piece sampling connector unit for use with a sample tube receiver.

### BACKGROUND OF THE INVENTION

Methods and devices for obtaining blood or blood component samples from a donor are well known. One such device employs a sample tube or vial and a sample tube receiver (or holder). The sample tube receiver is very typically a cylindrical barrel (or other shaped receptacle) with a piercing needle disposed inside the barrel and a luer or other tubular member extending from the distal end of the cylindrical barrel. The barrel of the receiver has a wide opening at its proximal end for receiving the blood sample tube. The tube or vial includes a rubber cap that is pierceable by the needle inside the sample tube holder barrel.

The luer end of the receiver may be permanently attached to a port on a blood tubing set in proximity to the blood flow path, as shown, for example, in U.S. Pat. No. 6,387,086. Alternatively, the luer end of the barrel may be fitted with a cannula (or other piercing member) for piercing an access site associated with a blood tubing set and/or a container in which blood or a blood component has been collected.

The latter is commonly used in connection with blood donations and blood processing performed at blood centers, hospitals and/or centers where blood apheresis is performed. One specific, but non-limiting example of such a system and device is the PlasmaLink® Sampling System available from Baxter Healthcare Corporation, of Deerfield, Ill., the assignee of the present application. The PlasmaLink® Sampling System is commonly used in obtaining samples of blood plasma collected from a donor using an automated apheresis device, such as the Autopheresis® C, also available from Baxter Healthcare Corporation, and generally described in U.S. Pat. No. 5,112,298. A blood component, separated from the whole blood of a donor, is collected in a container such as a bag or bottle. The container has associated with it a sampling access site. The access site is very typically a resilient, re-sealable septum which is preferably pre-slit. An example of such an access site is described in U.S. Pat. No. 5,188,620, which is incorporated by reference.

A system such as the PlasmaLink® Sampling System includes several components (which may be individually obtainable from different suppliers) which must be assembled by the technician. The components include a sample tube receiver, a sample tube adapter that includes a needle, a hub and a luer (hereinafter referred to as the “luer adapter”), and a separately provided blunt cannula for piercing the septum of the access site. The luer adapter is attached to one end of the sample receiver and the blunt cannula is fitted onto the end of the luer portion of the luer adapter, thus, resulting in the finished sampling device. The cannula end of the assembled system is then inserted into the access site of the container and an evacuated sample tube or vial is inserted into the barrel of the receiver where the rubber cap is pierced by the needle inside the barrel. The desired blood or blood component flows from the container into the tube and a sample is collected.

While this system and other analogous systems have worked satisfactorily, providers of healthcare products continue to look for ways to improve the efficiency and safety of blood sampling (and blood collection, generally) at a reduced cost to the medical community.

One improvement to presently used sampling systems would be to provide a sampling system that reduces the number of assembly steps required by the technician. Another improvement would be to provide a sampling system wherein the risk of improper cannula attachment (which could result in blood leakage or disassociation of the cannula from the luer) is reduced. Yet another improvement would be to provide a sampling system where the amount of waste from used parts (such as the end caps used to sheath the multiple parts) is reduced. From the consumer's standpoint, it would also be desirable, if a single, integrated connector unit were available from a single manufacturer. These and other objectives are addressed by the present invention.

### SUMMARY OF THE INVENTION

In one aspect, the present invention is directed to a one-piece connector and hub sub-unit for attachment to a barrel of a sample tube receiver. The sub-unit includes a first end, a second end, and a central portion between the first and second ends, which is adapted for attachment to the barrel, such that the second end is located within the barrel. The first end defines a first piercing member terminating in a blunted end. The second end is adapted for receiving a second piercing member. The sub-unit defines an internal flow path extending between the blunted end and the second end so as to allow for fluid communication between the blunted end and a second piercing member when received by the second end.

In another aspect, the present invention is directed to a one-piece, connector unit for attachment to the barrel of a sample tube receiver. The unit includes a first end, a second end, and a central portion between the first and second ends wherein the central portion is adapted for attachment to the barrel, such that the second end is located within the barrel. The unit further comprises a first end defining a first piercing member terminating in a blunted end and a second end defining a second piercing member. The unit defines an internal flow path extending between the first and second piercing members.

In a further aspect, the present invention is directed to a sampling device that includes a sample tube receiver comprising a barrel having a distal end and an open proximal end for receiving a sample tube. The sampling devices include a one-piece connector unit and needle assembly for attachment to the distal end of the barrel. The one-piece unit includes a first end, a second end and a central portion between said first and second ends. The central portion is adapted for attachment to the barrel such that the second end is located within the barrel. The unit further includes a first end defining a first piercing member and a second end defining a second and piercing member. The unit defines an internal flow path extending between the first and second piercing members.

### BRIEF DESCRIPTION OF THE DRAWINGS

FIGS. 1A-1C are plan views depicting the several components and assembly steps of a known sampling device and method for assembling the same;

FIG. 2A is a plan view of a fully-capped, one-piece connector unit embodying the present invention;

FIG. 2B is a plan view of the connector unit embodying the present invention with a first end cap removed;

FIG. 2C is a plan view of the connector unit embodying the present invention attached to a sample tube receiver, and a removed second end cap;

FIG. 2D is an end view of the second end cap of FIG. 2C;

FIG. 3 is a plan view of a blood collection container with sampling access site, a sample tube and a sample tube receiver with a connector unit embodying the present invention;

FIG. 4 is a plan view of a sample tube receiver with a connector unit embodying the present invention attached thereto;

FIG. 5 is an exploded view of a sample tube receiver and the connector unit embodying the present invention;

FIG. 6 is a plan view of a connector and hub subunit embodying the present invention;

FIG. 7 is an end view of the connector and hub subunit of FIG. 6;

FIG. 8 is a cross-sectional view of the connector and hub sub-unit of FIG. 6;

FIG. 9 is a cross-sectional end view of the connector and hub sub-unit of FIG. 8.

### DETAILED DESCRIPTION OF THE DRAWINGS

Although the present invention finds particular application in the field of blood sample collection and is described below in the context of its preferred use, it will be understood and appreciated that the present invention is not limited to use in this field. The present invention may be used wherever there is a need to provide a one-piece, integrated connector unit and/or where, for example, it is desirable to provide a ready-to-use connector unit.

As previously described, blood samples are very often collected using a sample tube receiver (or holder) and an evacuated sample tube or vial. Evacuated sample tubes or vials are well-known. An example of a known sample tube is one sold under the name VACUTAINER®, available from the Becton-Dickson Company, of Franklin Lakes, N.J.

In blood sample collections using a blood sample tube and, for example, a VACUTAINER® or other similar sample tube, blood samples are typically withdrawn from a bag, bottle or other receptacle having a sampling access site. The blood or biological fluid is withdrawn through the luer end of the sample tube receiver and introduced into a sample tube, which has been inserted into the sample tube receiver through the large opening at the proximal end. As many sample tubes may be filled as necessary.

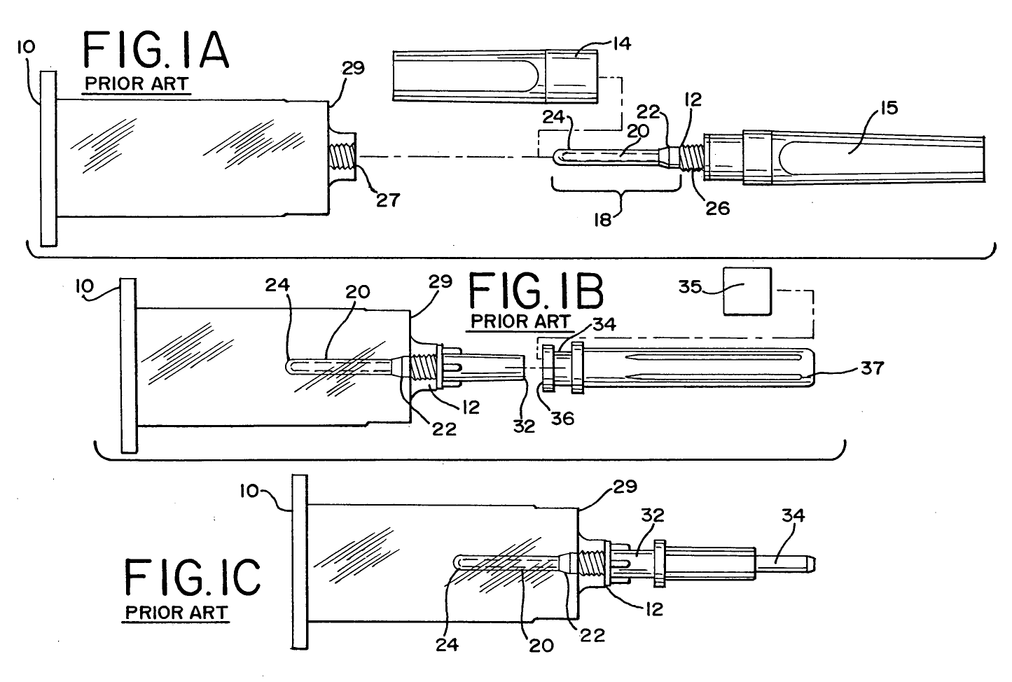

FIGS. 1A-1C show the several components and assembly steps of a known sampling device and the method for assembling the multi-component device. As shown in FIGS. 1A-1C, the medical technician practicing current sampling techniques requires a cylindrical, or other shaped, sample tube receiver 10 and a luer adapter 12. As shown, luer adapter 12 typically includes a sharpened stainless steel needle 20 secured onto mount or hub 22 of luer adapter 12. Typically, needle 20 is enclosed within a rubber sleeve 24, which prevents leakage of blood into the barrel of the receiver 10. The luer adapter 12 is initially enclosed in a two-part sheath or caps 14 and 15. Removal of end cap 14 exposes the needle 20 end of the luer adapter 12.

As shown in FIG. 1A, luer adapter 12 includes an externally threaded region 26 which allows the luer adapter 12 to be screwed into internally threaded region 27 at distal end 29 of holder 10. During assembly of the sampling device, the technician holds end cap 15 and screws the luer adapter 12 into the distal end 29. When luer adapter 12 has been firmly secured to holder 10, end cap 15 may be removed by further twisting cap 15 to expose luer end 32 of adapter 12.

To complete the assembly of the device, luer end 32 must be provided with a piercing member to allow for penetration of the access site of the container from which samples are to be drawn. Accordingly, a separately provided cannula 34 or other piercing member is required to convert the luer end 32 into a piercing end capable of piercing the access site (such as a resealable septum) of a blood or blood component container. Cannula 34 is also typically provided with a pair of caps 35 and 37. Removal of cap 35 exposes end 36 of cannula 34. While holding the cannula 34 by cap 37, the open end of cannula 34 is press-fit onto luer end 32. Thus, the sampling system is assembled and is ready to be used to withdraw samples from the blood or blood component container.

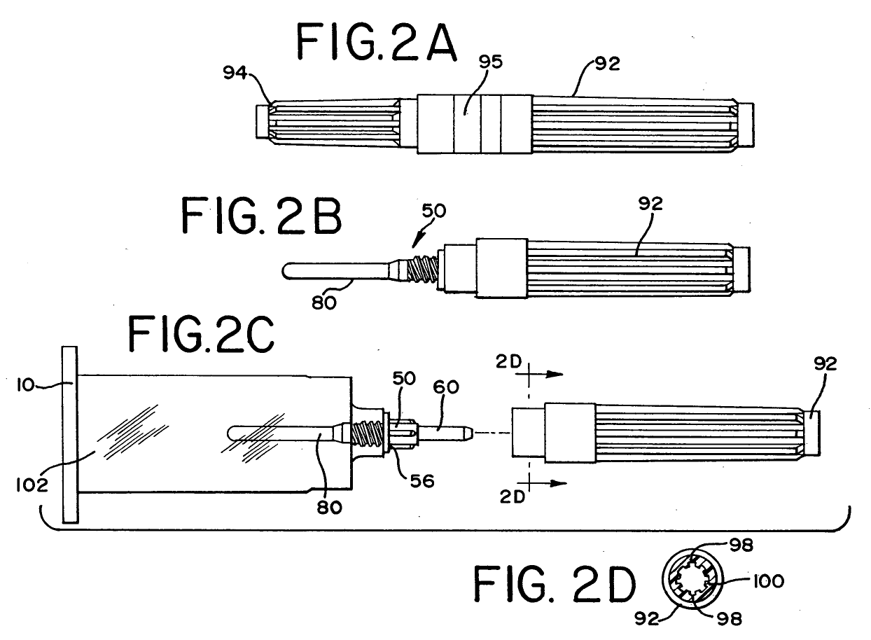

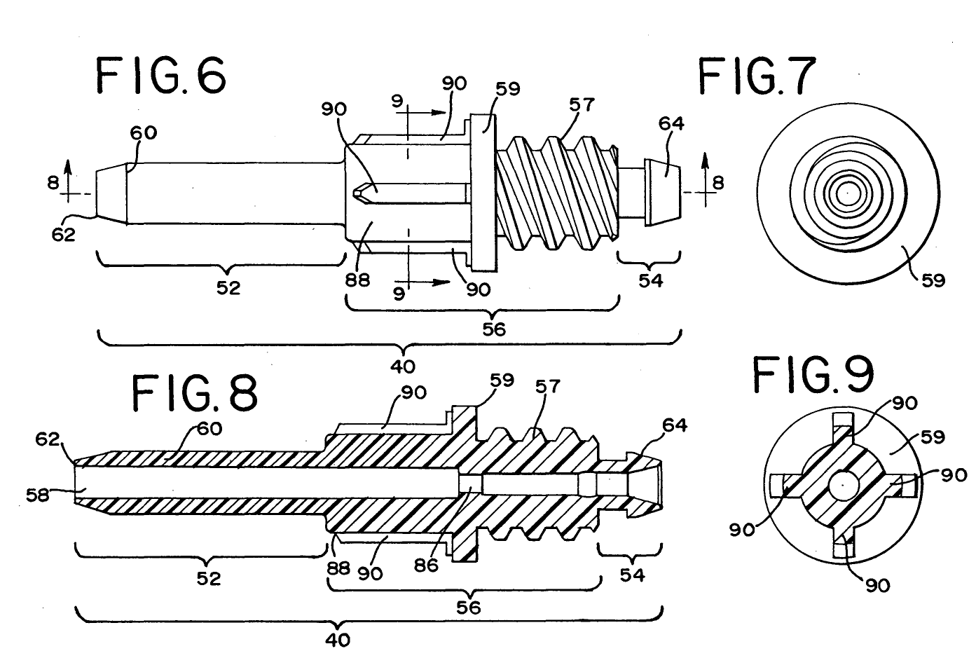

Using the present invention, some of the steps used in the above-described system can be eliminated. In FIGS. 2C, 4 and 5, there is shown a sample tube holder 10 (of the type shown and described above) with a one-piece connector unit 50 embodying the present invention associated therewith. As used herein, the term “connector unit” refers to the one-piece device that includes a first piercing member 60, a central portion 56 that includes a hub, and an oppositely facing piercing member 80 mounted on the hub. As used herein, the term “connector and hub sub-unit” refers to the portion of the connector unit 50 that includes a first piercing member and central portion 56 which is adapted to receive second piercing unit 80. The connector and hub sub-unit is designated by reference numeral 40 (and shown in FIGS. 6-9). Attachment of piercing member 80 to connector and hub sub-unit 40 results in connector unit 50.

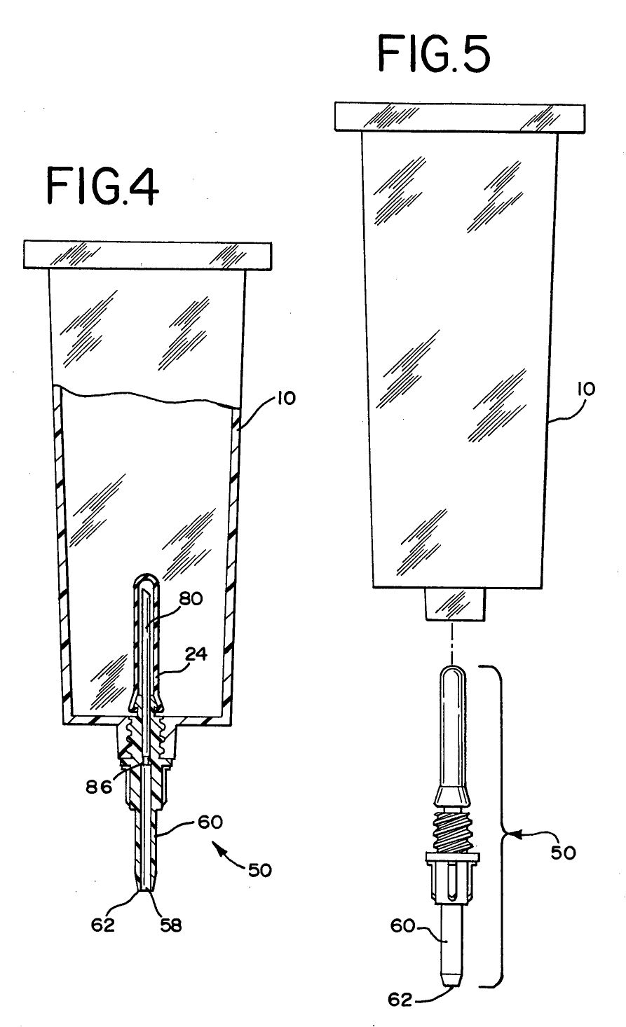

As shown in FIGS. 4 and 5, connector unit 50 is attached to the distal end of receiver 10. Connector unit 50 is a one-piece device with piercing member 80 pre-attached to subunit 40 (described in greater detail below.) Piercing member 80 includes a flexible sleeve 24 as will be known to those of skill in the art. Sleeve 24 may be made of any flexible material including rubber such as, but not limited to, latex.

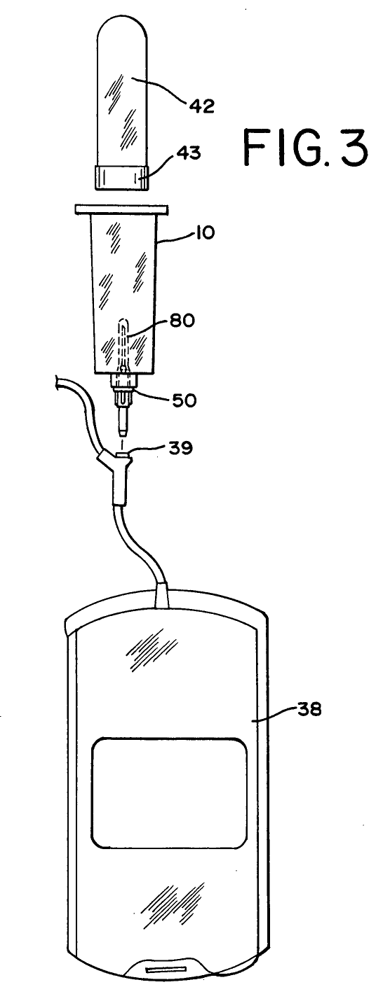

As shown in FIG. 3, a sampling device equipped with connector unit 50 of the present invention may be used to collect samples from a blood or blood component container 38 (such as a bag or bottle). Piercing member 60 is used to pierce sample access site 39 associated with container 38. Sample tube 42 is inserted into receiver 10 where cap 43 is pierced by piercing member 80, causing blood or the blood component to fill tube 42.

Turning now to FIGS. 6-9, connector and hub sub-unit 40 embodying the present invention includes a first end 52, a second end 54, and a central portion 56. In addition, as shown in the cross-sectional view of FIG. 8, the connector and hub sub-unit 40 includes an internal passageway 58, which establishes flow communication between the first end 52 and second end 54.

As further shown in the Figures, first end 52 defines first piercing member 60. Piercing member 60 may be a needle or a cannula. In a preferred embodiment, piercing member 60 is a cannula terminating in a blunt end 62. Although the dimensions and size of piercing member 60 may vary, in one embodiment, piercing member 60 has an outer diameter of approximately 2.0-3.0 mm. The length of piercing member 60 (or first end 52) may be between approximately 9.0-11.0 mm.

Second end 54 includes a needle mount 64 adapted to receive a second piercing member. Preferably, the second piercing member, when attached, is a hollow stainless steel needle 80 (best seen in FIG. 4) secured to mount 64 by epoxy or other adhesive. Alternatively, piercing member 80 may be press fit or otherwise friction fit into mount 64. In one embodiment, needle 80 may have a length of between approximately 22.0-23.1 mm, an outer diameter of approximately 0.860-0.920 mm, an inner diameter of at least approximately 0.560 mm. Of course, any of these dimensions may be varied as needed.

Connector and hub sub-unit 40 includes a central portion 56 between first end 52 and second end 54. At least a part of central portion 56 is adapted for attachment to distal end 29 of the barrel of holder 10. Preferably, central portion 56 includes annular external threads 57 that allow the sub-unit 40 (and, for that matter, connector unit 50) to be screwed into the distal end of holder 10.

Also, as shown in FIGS. 6 and 8, sub-unit 40 includes an annular collar 59 that has a diameter greater than the diameter of piercing member 60 and hub 88 (described below) of sub-unit 40. Collar 59 acts as a stop for the first end of the unit 40 when unit 50 is attached to holder 10.

Shown in FIG. 8 is a cross-sectional view of the sub-unit 40 embodying the present invention. As shown in FIG. 8, sub-unit 40 includes and defines an internal flow path 58 which extends from the first end 52 to second end 54. Internal flow path 58 provides flow communication between the ends of sub-unit 40. When sub-unit 40 has been provided with needle 80 at second end 54, resulting in a fully assembled and fully integrated connector unit 50, internal flow path 58 provides flow communication from one end of the first piercing member 60 to the end of the other piercing member 80.

As shown in FIG. 8, the diameter of internal flow path 58 may not be uniform and, in fact, may be narrower in the area of central portion 56. The narrowed portion 86 of the internal flow path 58 acts as a catch or stop for needle 80 when needle 80 is inserted through the second end of sub-unit 40. Accordingly, it is preferred that the diameter of portion 86 be smaller than the outer diameter of needle 80. Although the dimensions of the sub-unit may vary, depending on the use of sub-unit 40, in one embodiment, the diameter of internal flow path within first end 52 may be between 1.0-2.0 mm, with a preferred diameter of approximately 1.4 mm.

As further shown in FIGS. 6 and 8, in addition to threaded region and collar 59, central portion 56 of sub-unit 50 includes a hub 88 between the piercing member 60 and the collar 59. Hub 88 preferably includes a plurality of fins 90 radially spaced about hub 88. As described in greater detail below, fins 90 interact with slots 98 on the interior surface of end cap 92 (shown in FIG. 2D). Specifically, fins 90 are captured within slots 98, thereby securing cap 92 to sub-unit 40.

Sub-unit 40 is a one-piece unit that is preferably made of a biocompatible, thermoplastic material. The material used should be sterilizable by forms of sterilization suitable for thermoplastic medical products, such as treatment with ethylene oxide (ETO) or radiation sterilization, such as electron beam radiation and gamma radiation. A preferred material for the sub-unit 40 is polypropylene. Of course, other polymers and co-polymers that meet the requirements described above may also be used in making the sub-unit 40. Preferably sub-unit 40 is injection molded, but any method of making a single unitary one-piece, integrated sub-unit 40 may be employed.

Turning briefly to FIGS. 2A-2C, the method of employing the one-piece connector unit 50 of the present invention, and the method of assembling a sampling device will now be described.

As shown in FIG. 2, unit 50 may be in an enclosed sheath or receptacle. In a preferred embodiment, the receptacle is provided as two end caps 92 and 94. Each end cap 92 and 94 has a closed end and facing open ends, sealed together by a breakable tamper-evident band 95. Removal of end cap 94 exposes piercing member 80. While grasping end cap 92, unit 50 is screwed into the distal end 29 of holder 10. Further twisting of end cap 92 releases fins 90 from the slots 98 on the interior surface 100 of end cap 92, thereby releasing end cap 92 and exposing the first end 52 (cannula 60), hub 88 and flange 59 of unit 50. With end cap 92 removed, sampling device 102 is ready for use. End caps 92 and 94 may also be made of a sterilizable (by, for example, ETO or radiation sterilization including electron beam and gamma radiation) thermoplastic material and injection molded. As in the case of sub-unit 40, a preferred material for end caps 92 and 94 is polypropylene.

Using the one-piece connector unit 50 of the present invention, the technician can quickly assemble a sampling device with a cannula already in place. No separate attachment of a cannula member is required. In addition, fewer assembled parts results in less waste in terms of disposable end caps for the different pieces.

It will be appreciated that various modifications of the embodiments and methods described herein are possible, in accordance with the scope of the present invention.

That which is claimed:

1. A one-piece connector and hub sub-unit for attachment to the barrel of a sample tube receiver, said sub-unit comprising:
a first end, a second end, and a central portion between said first and second ends, said central portion being adapted for attachment to said barrel such that said second end is located within the barrel said first end defining a first piercing member terminating in a blunted end,
said second end adapted for receiving a second and separate piercing member,
said sub-unit defining an internal flow path extending between said blunted end and the second end so as to allow for fluid communication between said blunted end and a second piercing member when received by said second end.

2. The sub-unit of claim 1 further comprising a second piercing member attached to said unit at said second end, said second piercing member comprising comprises a hollow needle.

3. The sub-unit of claim 1 wherein said unit is molded from a thermoplastic material.

4. The sub-unit of claim 1 wherein said thermoplastic material comprises polypropylene.

5. The sub-unit of claim 1 wherein said central portion comprises annular threads for engagement with said barrel.

6. The sub-unit of claim 2 comprising a flexible sleeve over said needle.

7. The sub-unit of claim 2 further comprising a pair of caps enclosing said first and second piercing members.

8. A one-piece connector unit for attachment to the barrel of a sample tube receiver, said unit comprising:
a first end, a second end, and a central portion between said first and second ends, said central portion being adapted for attachment to said barrel such that said second end is located within the barrel
said first end defining first piercing member terminating in a blunted end,
said second end defining a second and separate piercing member,
said unit defining an internal flow path extending between said first and second piercing members;

9. The unit of claim 8 further comprising:
a removable cover enclosing said first piercing member; and
a removable cover enclosing said second piercing member.

10. The unit of claim 9 wherein each of said covers comprises an open and a closed end, wherein said open ends face one another.

11. The unit of claim 9 wherein said first end comprises a hub including a plurality of radial fins thereon and said removable cover enclosing said first piercing member comprising an inner wall, said inner wall including a plurality of slots for receiving said fins.

12. A sample tube receiver assembly comprising:
a barrel having a open proximal end for receiving a sample tube, and a distal end;
a one-piece connector unit for attachment at said distal end of said barrel;
said unit comprising a first end, a second end, and a central portion between said first and second ends, said central portion being adapted for attachment to said barrel such that said second end is located within the barrel;
said first end defining first piercing member, said second end defining a second and separate piercing member,
said unit defining an internal flow path extending between said first and second piercing members.

13. The assembly of claim 12 wherein said first piercing member terminates in a blunted end.
